(19) United States
(12) Patent Application Publication
(10) Pub. No.: US 2025/0318812 A1
(43) Pub. Date: Oct. 16, 2025

(54) NOUVELLE COMFORT PAPANICOLAOU (PAP) BRUSH

(71) Applicant: Steven D. Broman, Fort Collins, CO (US)

(72) Inventor: Steven D. Broman, Fort Collins, CO (US)

(21) Appl. No.: 19/178,209

(22) Filed: Apr. 14, 2025

Related U.S. Application Data
(60) Provisional application No. 63/633,754, filed on Apr. 13, 2024.

(51) Int. Cl. A61B 10/02

(52) U.S. Cl. CPC A61B 10/02; A61B 2010/0216

(57) ABSTRACT

A cytological collection tool comprising a handle attached at its distal end to a base that includes a head from which broom-like tubules protrude and are arranged to form an elongated central portion and from which an arrangement of brush-like fibers protrude from the central portion.

### CROSS-REFERENCE TO RELATED PATENT APPLICATIONS

This application claims priority to and the benefit of U.S. Provisional Patent Application Ser. No. 63/633,754, filed Apr. 13, 2024, which is incorporated herein in its entirety by reference.

### FIELD OF THE INVENTION

This invention relates to gynecological medical devices, in particular cytological collection tools utilized for the acquisition of tissue samples during pelvic examinations.

### BACKGROUND OF THE INVENTION

The United States Preventative Services Task Force (USPSTF) makes recommendations for cancer screenings for five common cancers (breast, colon, prostate, lung, and cervix), which comprise the standard of appropriate preventative medical care in the U.S. today. Frequent screening results in earlier detection and treatment of these diseases, resulting in potential cures, improving quality and quantity of life for patients.

The uterine cervix is the inferior entrance into the uterine cavity and is shaped like a miniature donut. The cells most likely to transform into malignancies are located in the donut's hole, extending back out onto the distal surface. For almost a century, the Papanicolau (Pap) smear technique has been utilized to evaluate the cells from the examination of the female cervix for almost a century.

To facilitate the pelvic exam and acquire the cervical cells for evaluation, the vaginal speculum has been instrumental. Currently, most physician offices that perform pap smears are supplied with three different sizes of speculums for proper visualization of the female anatomy and collection of cervical cells. Skilled physicians are keenly aware of the techniques required (proper speculum choice, instrument lubrication, manual insertion procedures, etc.) to minimize patient discomfort and subsequent reluctance to repeat testing. The choice of speculum size is crucial in mitigation of patient discomfort but the current choices available also have drawbacks. Many clinicians use the medium sized speculum for better visualization of the cervix and side walls as well as for the easy utilization of the most common cell samplers currently in use. Many other clinicians primarily use the smaller speculums for most pelvic examinations for increased patient comfort. Utilizing a smaller speculum, however, still often results in significant patient discomfort during the procedure because of the current larger instruments available that are utilized for cell sample acquisition.

Currently, the initial step in obtaining cervical cells for examination utilizes a plastic broom to collect cells and other material from a large part of the central anterior/front surface of the cervix. The second step in obtaining cervical cells for examination insures even greater capture of potentially cancerous cells by utilizing a nylon brush specifically to obtain cells from the donut hole (endocervix) itself where more malignant cell transitions tend to occur. After each of these collections capture cells and other materials, they are removed and rinsed with a preservative fluid that is later evaluated microscopically for abnormal cells.

The current two-step process is complimentary; i.e. abnormal cells located on the distal or front surface can be easily sampled with a broad plastic broom, but the more malignant-prone cells located in the donut hole (endocervix) are more efficiently sampled with a nylon brush. As a result, most clinicians utilize both steps to effectively screen for cervical cancer.

The current procedures for obtaining cervical cells in the field suffer from several drawbacks. First, the current procedure often results in unnecessary patient discomfort due to the use of larger than needed vaginal speculums to accommodate the current large specimen samplers. Similarly, the additional time required to complete the cell acquisition in a two-step method also often results in additional patient discomfort and anxiety. This patient discomfort and anxiety create a suboptimal experience for a medically necessary procedure that often results in a hesitancy to schedule future screenings. As a result, there can be a delay in early cancer detection and treatment. Further, clinician anxiety is generated because of empathy for the patient during such sensitive procedures and all good, caring providers want to create an experience that is as close to neutral in an uncomfortable but necessary setting. Prior art attempts to create a single-step collection tool include plastic brooms with a longer central bristle portion. However, these designs are less effective at collecting cells from within the endocervix. Other prior art attempts to create a single step collection tool include tapered nylon brushes. While these designs may be effective at collecting cells from the endocervix region, the designs fail to adequately sample cells from the anterior portion of the cervix.

Accordingly, a need exists for a novel medical device to screen for cervical cancer and other intravaginal lesions that collects sufficient samples from both the endocervical region and anterior portion of the cervix (or portio) in a single-step to minimize patient discomfort and its resulting patient and clinician anxiety.

### SUMMARY OF THE INVENTION

The novel apparatus disclosed herein overcomes the drawbacks of the prior art by utilizing a smaller collection tool comprising both a narrower broom and nylon brush that allows collection of cells for testing in a single step for histological analysis.

### BRIEF DESCRIPTION OF THE DRAWINGS

FIG. 1 illustrates an exemplary embodiment of a prior art plastic broom.

FIG. 2 illustrates an exemplary embodiment of a prior art nylon brush.

FIG. 3 is an exemplary embodiment of the invention described herein.

FIGS. 4A-4B are an exemplary embodiment of the invention described herein.

In the drawings like characters of reference indicate corresponding parts in the different figures. The drawing figures, elements and other depictions should be understood as being interchangeable and may be combined, modified, and/or optimized in any like manner in accordance with the disclosures and objectives recited herein as would be understood to those skilled in the art.

### DETAILED DESCRIPTION OF THE INVENTION

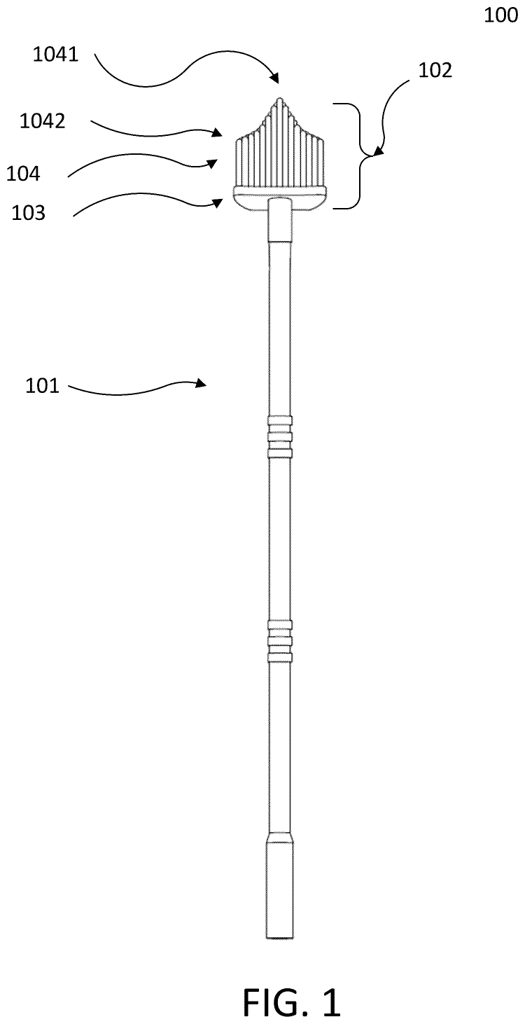

FIG. 1 illustrates a prior art plastic broom collection tool often utilized to collect superficial cells, mucus, and other debris from a large part of the central anterior/front surface of the cervix, comprising a length n and a width w. Such prior art broom collection tools include the Wallach Papette Cervical Cell Collector Brush, Rovers Cervex-Brush, and similar designs well-known to one of ordinary skill in the art.

The prior art plastic broom collection tool 100 is comprised of a handle 101, utilized by the physician to insert and manipulate the tool, and a broom head 102 comprised of plastic tubules 104 of varying length protruding from base 103 and arranged to form an elongated central portion 1041 that protrudes from edge portions 1042 located at the distal end of the tool. One of ordinary skill in the art would recognize that prior art broom collection tools are available in different lengths and broom widths to better accommodate the anatomy of the patient. After insertion of a speculum sufficiently large to accommodate the broom's width, the physician utilizes the broom 100 to complete one or more circumferential sweeps, e.g., 1-5, of the anterior surface of the cervix and then removes the broom 100 with superficial cells collected by the sweep(s). The current designs of broom head 102, with the elongated central portion 1041 and edge portions 1042, allows the collection of superficial cells from both the external surface of the cervix as well some amount of superficial cells from its center hole (called the endocervix). While prior art plastic broom collection tools may comprise a central portion 1041 in which the bristles of the broom are longer, such lengthier bristles often are incapable of collecting sufficient superficial cells from the endocervix. The broom 100 is then removed from the patient and rinsed with a preservative fluid, which is later evaluated microscopically for abnormal cells.

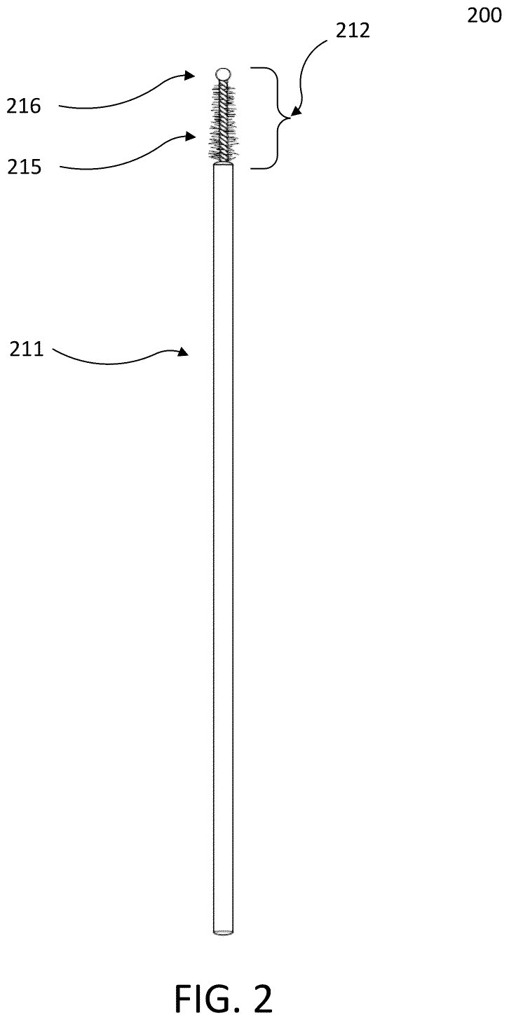

FIG. 2 illustrates a prior art tapered nylon brush collection tool 200 often utilized as a second step in a cervical examination to collect cells from within the endocervix itself where more malignant cell transitions tend to occur. The prior art nylon brush tool 200 is comprised of a handle 211, utilized by the physician to insert and manipulate the tool, and a brush head 212 comprised of a conical arrangement of soft nylon fibers 215 topped with a protective tip 216 located at the distal end of the brush tool 200. One of ordinary skill in the art would recognize that prior art brush collection tools are available in different lengths and brush widths to better accommodate the anatomy of the patient. After removal of the plastic broom tool 100, the nylon brush tool 200 is then inserted into the patient to better collect superficial cells from the interior region of the endocervix. After its collection of superficial cells, mucus and debris from the endocervix area, the nylon brush 200 is removed and rinsed with a preservative fluid that is, similarly, later evaluated microscopically for abnormal cells. Such prior art brush collection tools include the Puritan Histobrush and similar designs well-known to one of ordinary skill in the art.

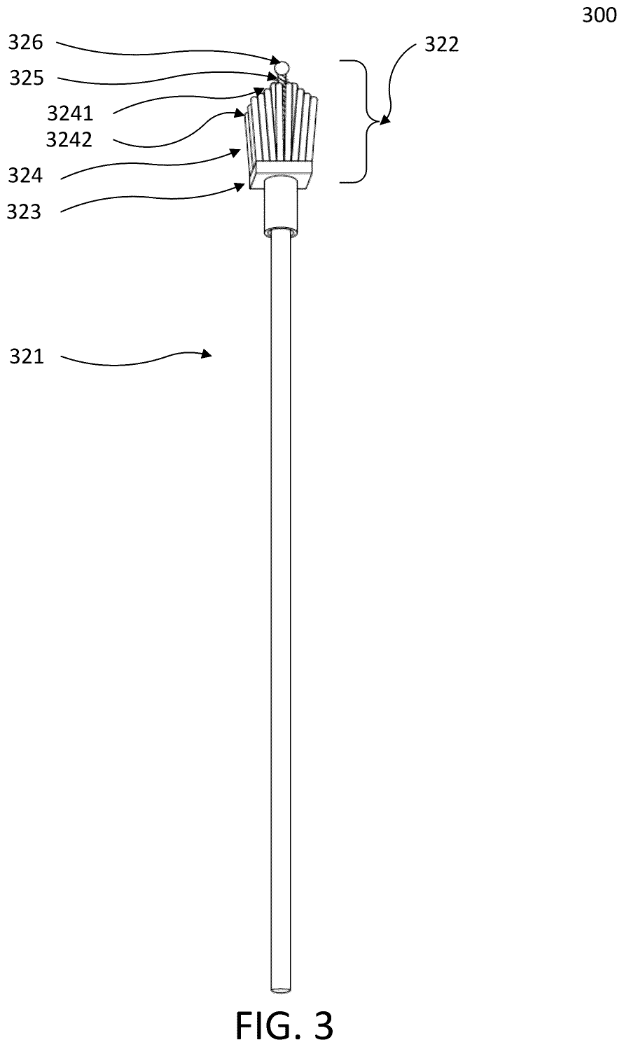

FIG. 3 illustrates an exemplary embodiment of the invention disclosed herein. The embodiment disclosed in FIG. 3 is an improved cytological collection tool that allows the collection of superficial cells from both the exterior region of the cervix and from the endocervix in a single step. The collection tool 300 of this embodiment is comprised of a handle 321, utilized by the physician to insert and manipulate the tool, and a head 322 comprised of plastic tubules 324 of varying length protruding from base 323 and arranged to form an elongated central portion 3241 that protrudes past edge portions 3242 located at the distal end of the tool. In addition, protruding from the central portion 3241 is a conical arrangement of soft nylon fibers 325 topped with a protective tip 326 located at the distal end of the brush tool 300. Tubules 324 are more flexible than the nylon fibers 325.

One of ordinary skill in the art would recognize that prior art brush collection tools are available in different lengths and brush widths to better accommodate the anatomy of the patient. Given the arrangement of the plastic tubules 324 and the conical arrangement of soft nylon fibers 325, one of ordinary skill in the art would recognize that the width of base 323 may be substantially reduced from the width of base 103 in prior art broom 100, allowing for the selection of a smaller speculum during the procedure that increases the comfort of the patient during the procedure.

In a further embodiment, the edges of base 323 can be modified through a bevel, chamfer, rounding, or fillet to increase patient comfort.

In a further embodiment, the corners of base 323 can be modified by rounding.

In a further embodiment, plastic tubules 324 may be arranged at a uniform height from which a conical arrangement of soft nylon fibers 325 topped with a protective tip 326 protrudes.

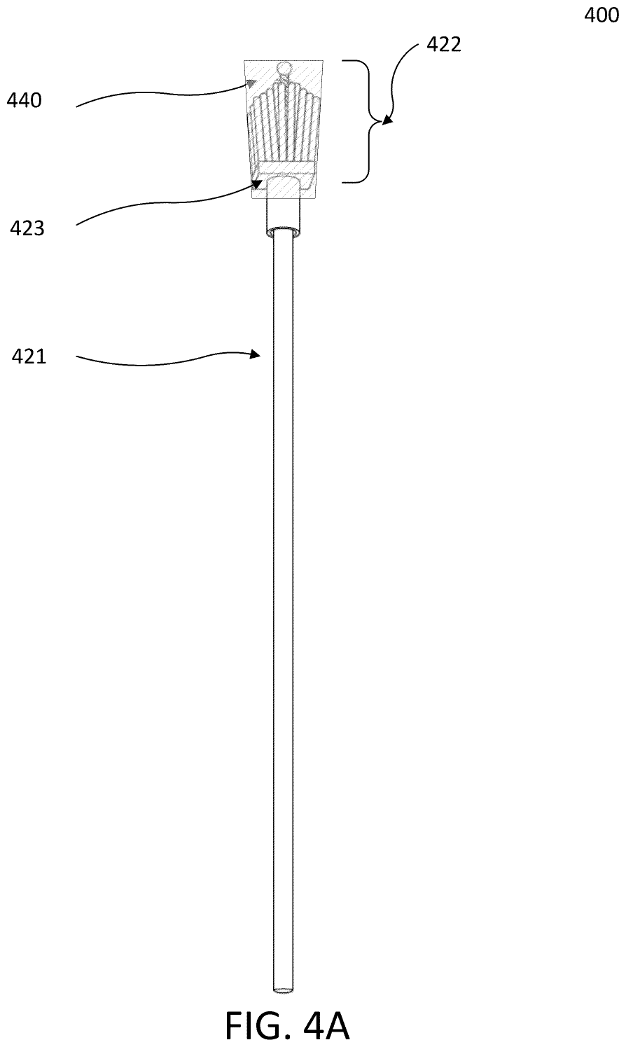

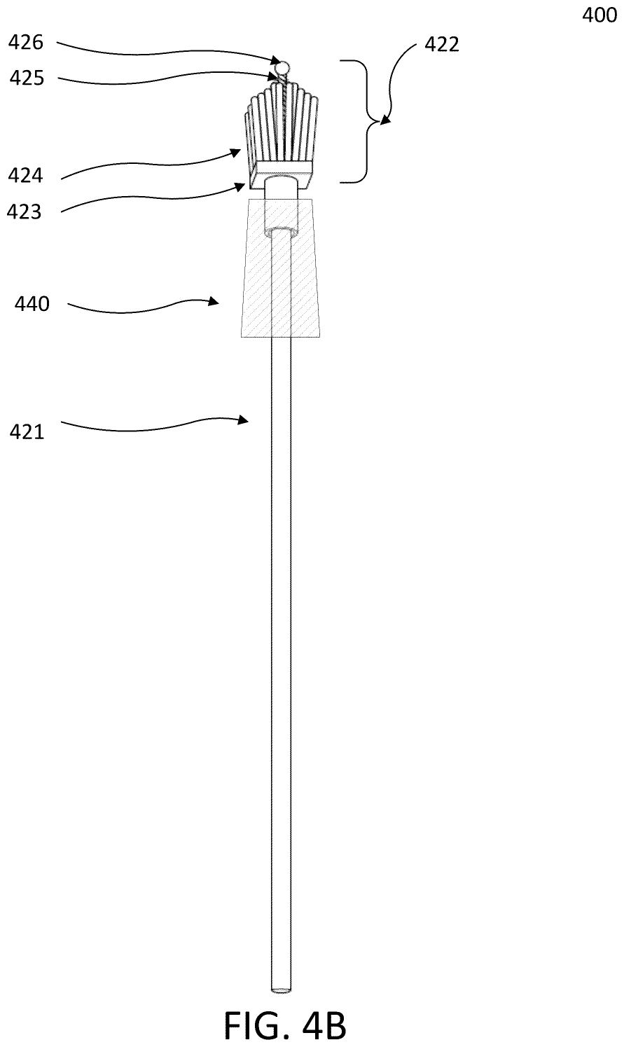

In a further embodiment, shown in FIGS. 4A and 4B, the collection tool 400 may include a moveable sheath 440, composed of a thin film of pliable material that is moveable from a distal position, in which the moveable sheath 440 surrounds base 423 as shown in FIG. 4A, and a proximal position, in which the moveable sheath is moved along handle 421 to expose plastic tubules 424, soft nylon fibers 425 and protective tip 426 the base 423 as shown in FIG. 4B. When inserted, the moveable sheath 440 is in a position to protect head 422 from contamination. Once the tool 400 is in position for collection of superficial cells, the moveable sheath 440 is retracted, exposing head 422 such that the plastic tubules 424 and soft nylon fibers 425 are in contact with the cervix and endocervix, respectively, for collection of superficial cells. Once the collection is completed but prior to beginning the removal of collection tool 400 from the patient, moveable sheath 440 is re-extended to again protect head 422 and the superficial cells collected by plastic tubules 424 and soft nylon fibers 425 from potential contamination. The moveable sheath 440 may be retracted and returned to position by mechanical structures in the handle or through other means known to one skilled in the art.

In a further embodiment, the moveable sheath 440 is comprised of a thin sheet of the same material as plastic tubules 424.

In a further embodiment, the moveable sheath transits along handle 421 from a position covering the head 422 to a position in which head 422 is exposed.

This present invention disclosure and exemplary embodiments are meant for the purpose of illustration and description. The invention is not intended to be limited to the details shown. Rather, various modifications in the illustrative and descriptive details, and embodiments may be made by someone skilled in the art. These modifications may be made in the details within the scope and range of equivalents of the claims without departing from the scope and spirit of the several interrelated embodiments of the present invention.

What is claimed is:

1. A cytological collection tool, comprising:
a handle;
a base located at the distal end of the handle;
a head that protrudes in the distal direction from the handle;
wherein the head is comprised of;
tubules protruding in the distal direction from the base and arranged to form a central portion that protrudes further from the handle than the edge portions;
an arrangement of fibers located near the center of the central portion of the tubules;
wherein the tubules are more flexible than the fibers; and
a protective tip topping the arrangement of fibers.

2. The cytological collection tool of claim 1, wherein the base of the tool is covered by a moveable sheath that protects the head from contamination during insertion and removal of the tool which sheath is moveable to expose the head during collection.

3. The cytological collection tool of claim 1, wherein the tubules are manufactured from a thermoplastic elastomer.

4. The cytological collection tool of claim 2, wherein the tubules are manufactured from a thermoplastic elastomer.

5. The cytological collection tool of claim 1, wherein the tubules are manufactured from an isomannide-based linear polymer.

6. The cytological collection tool of claim 2, wherein the tubules are manufactured from an isomannide-based linear polymer.

7. The cytological collection tool of claim 1, wherein the fibers are arranged in a conical shape.

8. The cytological collection tool of claim 2, wherein the fibers are arranged in a conical shape.

9. The cytological collection tool of claim 1, wherein the fibers are manufactured from a thermoplastic polyamide.

10. The cytological collection tool of claim 2, wherein the fibers are manufactured from a thermoplastic polyamide.

11. The cytological collection tool of claim 1, wherein the fibers are manufactured from a thermoplastic polyolefin.

12. The cytological collection tool of claim 2, wherein the fibers are manufactured from a thermoplastic polyolefin.
